(19) United States
(12) Patent Application Publication
(10) Pub. No.: US 2005/0287032 A1
(43) Pub. Date: Dec. 29, 2005

(54) Anti-bacterial protection to improve performance of post CMP clean brush

(76) Inventors: Alexander Tregub, Oak Park, CA (US); Mansour Moinpour, San Jose, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/877,561

(22) Filed: Jun. 24, 2004

(51) Int. Cl. A61L 2/16

(52) U.S. Cl. 422/1; 422/24; 422/28; 424/411

(57) ABSTRACT

A method including providing a residual bacteria-inhibiting property to a post-chemical mechanical polish (post-CMP) brush. A method including forming a post-chemical mechanical polish (post-CMP) brush; and residually modifying a bacteria-inhibiting property of a portion of an exterior surface of the formed brush. An apparatus including a post-chemical mechanical polish (post-CMP) brush including a residual bacteria inhibiting property.

### BACKGROUND

1. Field

Substrate cleaning.

2. Background

Chemical mechanical polishing (“CMP”) processes are widely used in manufacturing of semiconductor circuits for planarization and/or removing excess of dielectric and metal materials. Many commercially available rotary and orbital polishers may be used to perform a CMP process. One example is the Reflexion™300 mm CMP system, which is available from Applied Materials, Inc.

A chemical mechanical process involves application of a polymer-based polishing pad(s) and abrasive slurries. After the CMP process is completed, a substrate (e.g., wafer) may be covered with residuals of abrasives, pads, or polished out metal/oxide layers. Residual slurry and polished out layers should be removed to avoid such particles/contaminants inhibiting subsequent processing. To remove particles/contaminants, the substrate may be subjected to one or a series of cleaning and scrubbing operations. Many commercially available tools, e.g., the Reflexion system mentioned above, integrate CMP modules with the post CMP cleaning modules that perform those cleaning and scrubbing functions. Such tools may include one or more high porosity foam (such as poly vinyl (alcohol) (“PVA”)) brushes, for example, included in a brush box. In the presence of deionized water or a dilute cleaning agent rinse, such brush(es) contact a surface of the substrate and remove the particles/contaminants through mechanical action. An additional cleaning technique includes sonic or ultrasonic cleaning that may be used in conjunction with a brush treatment.

One current technology trend is the substitution of traditional dielectric materials such as silicon dioxide with a dielectric material having a dielectric constant less than silicon dioxide (a “low-k” dielectric material). Certain materials that may be used to form low-k dielectric layers (e.g., carbon doped oxides) may have surface energies different than traditional silicon dioxide. Post-CMP cleaning solutions may seek to match these surface energies. Current brushes, however, do not provide capabilities to adjust their surface energies to make it close to that of the cleaning solution to effectively clean a substrate. In other words, surface energy synergism in the system solution-brush-substrate to improve effectiveness of the substrate cleaning cannot be achieved.

Bacteria growth in polymer materials that constitute the working portion of a post-CMP brush can dramatically change physical and mechanical properties of the brush, such as compressive stress, or destroy the polymer after prolonged storage. Currently, post-CMP brushes from different suppliers are shipped in sealed bags with a wet chemical (transient) anti-bacterial treatment. Typical wet chemical treatments include ammonium hydroxide, or oxalic acid, or other treatments. A typical wet chemical treatment involves dipping the brush in the chemical and then removing the excess (e.g., squeezing out the excess) and sealing the brush in a bag. A major problem with wet treatments is that a polymer material of the brush can be degraded when exposed to a liquid ambient, such as water, ammonium hydroxide, oxalic acid. Those changes occur due to polymer plastisizing that affect mechanical properties. Moreover, these changes depend on brush storage time, temperature conditions, other external factors, and such. Another problem is that the amount of wet chemical (e.g., ammonium hydroxide, oxalic acid) is not quantified and therefore the excess chemical tends to change the properties of the polymer of the brush in an uncontrolled and unpredictable manner.

### BRIEF DESCRIPTION OF THE DRAWINGS

Features, aspects, and advantages of embodiments will become more thoroughly apparent from the following detailed description, appended claims, and accompanying drawings in which:

FIG. 1 shows a schematic side view of a dual roller post-CMP brush system and a substrate between the brushes.

FIG. 2 shows a top perspective side view of a knobby brush suitable for post-CMP cleaning processes.

FIG. 3 shows a top perspective side view of a ridged brush suitable for post-CMP cleaning processes.

FIG. 4 shows a flow chart describing various processes to impart a residual bacteria-inhibiting property to a post-CMP brush.

### DETAILED DESCRIPTION

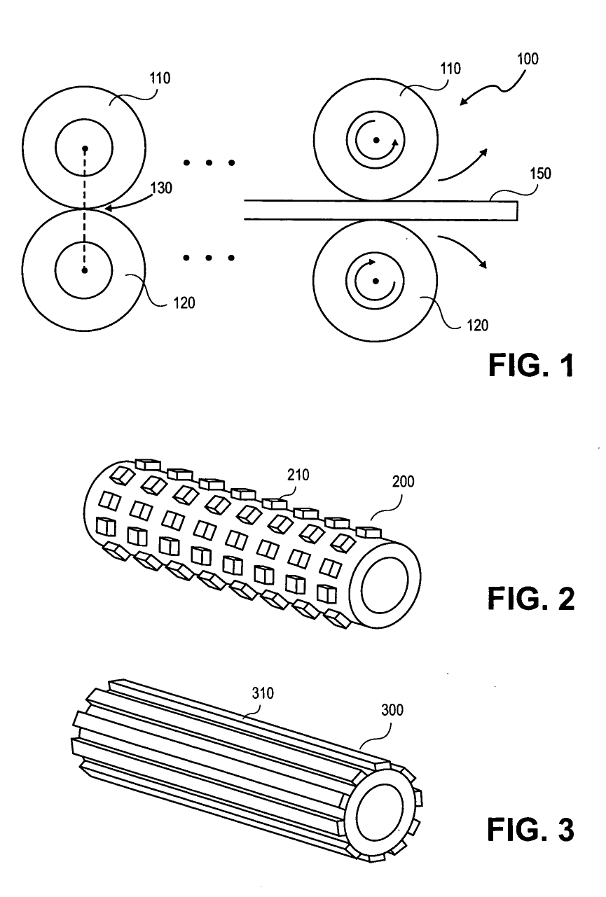

FIG. 1 shows a schematic side view of a typical dual roller post-CMP brush system. System 100 includes roller brush 110 and roller brush 120. Each brush is a tubular structure that is capable of being rotated on a spindle. In this embodiment, brush 110 and brush 120 have axes vertically aligned (as viewed) and the sides of the brushes contact one another at contact point 130. Moving a wafer through contact point 130 separates brush 110 from brush 120 and rotation of the brushes provides scrubbing action to a wafer.

Brush 110 and brush 120 are made of a relatively soft, porous foam polymer such as poly vinyl (alcohol) (PVA). FIG. 2 and FIG. 3 show two types of brushes. FIG. 2 shows a top perspective side view of a knobby brush suitable for post-CMP cleaning processes. FIG. 2 shows knobby brush geometry 200 in which an outer surface includes protruding cylinders 210 raised at right angles to the curved brush surface. FIG. 3 shows a ridged (splined) brush suitable for post-CMP cleaning processes. Ridged (splined) brush geometry 300 has a series of continuous raised strips 310 at right angles to the curved roller surface of the brush and parallel to its axis.

Referring again to FIG. 1, a typical brush, such as brush 110 and brush 120, has a length suitable to contact all portions of a side of a wafer. In other words, a length in one embodiment is equal or greater than a diameter of a wafer. A representative length is on the order of 10 to 15 inches (25 to 38 centimeters). Brush 110 and brush 120 have an outside diameter on the order of 2.25 inches (5.7 cm) and an interior diameter on the order of 1.25 inches (3.2 cm). Each brush is rotated on a spindle through which water or other cleaning solution may be pumped to saturate a porous polymer material of brush 110 and brush 120.

FIG. 1 shows wafer 150 moving through brush 110 and brush 120 during a post-CMP cleaning operation in system 100. As shown, brush 110 and brush 120 each rotate on their respective axis and contact opposite sides of wafer 150. The contact with wafer 150 dislodges and drives off particles. Although not shown, water or other cleaning solution separates the brushes from contact with each other and may be incorporated in system 100 and sprayed from nozzles onto wafer 150 from above and/or below.

As noted above, a typical material for a cleaning brush in a post-CMP system is a foam polymer such as PVA. PVA is known to be susceptible to biodegradation. Current methods to minimize biodegradation include storing a brush in transient chemical solutions such as ammonia or oxalic acid. These chemical solutions, however, tend to affect mechanical properties of a brush such as compressive stress. Additionally, the amount of time a brush is stored in a chemical solution may affect the mechanical properties of a brush leading to an unpredictable change of brush properties.

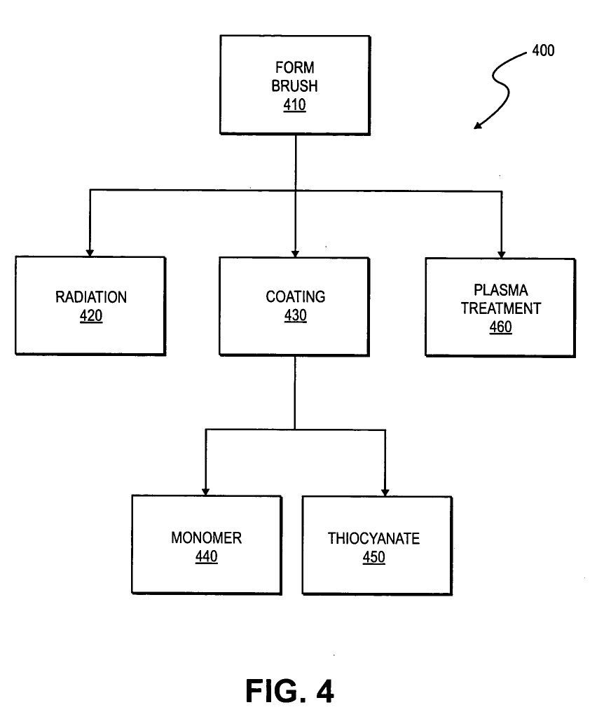

FIG. 4 shows a flow chart describing various methods to impart a residual bacteria-inhibiting property to a post-CMP brush. The various methods described in process flow 400 provide a residual bacteria-inhibiting property to a brush. These methods are contrasted with the approach of chemical solutions described in the past. By a residual property is meant that after such treatment/application, a bacteria-inhibiting property remains with the brush. For example, various techniques presented herein beneficially modify the surface of a brush to impart an anti-bacterial property to the brush. This is contrasted with the prior art approach of wet chemical solutions where the bacteria inhibiting effect is produced by the chemical solution and once the solution is removed, the susceptibility of the brush to bacteria contamination is the same as it was before the chemical solution treatment. Thus, the wet chemical treatments are referred to as transient in the sense that although such treatments reduce bacteria contamination, no beneficial property is imparted to the brush.

Referring to process flow 400, at block 410 a post-CMP cleaning brush is formed. In one embodiment, a brush is formed of a relatively soft and porous PVA material as known in the art. The brush may be either of the knobby type or ridged (splined) type or other configurations.

Once a brush is formed, the brush may be treated to impart a residual bacteria-inhibiting property to the brush. Such treatment may be done by the brush manufacturer or other entity. Referring to block 420, in a first process, a polymer brush may be radiated with, for example, an ultraviolet (UV) radiation source. The UV radiation penetrates the polymer and essentially sterilizes the brush. In one embodiment, a brush may be exposed to UV radiation for a period of seconds (e.g., 10-20 seconds) to modify the brush. One application of ultraviolet radiation to inhibit bacteria is described in “Bioactive assessment and bacteria test for the varied degrees of ultraviolet radiation onto the collagen-immobilized polypropylene non-woven fabric” by J. D. Liao and Y. C. Tyan, Biomedical Engineering Application Basis Communication, v. 4, no. 1 2002, pgs. 20-30.

Block 430 shows another technique to impart a residual bacteria-inhibiting property to a post-CMP brush. In this technique, a coating is applied to the brush surface. One type of coating is chemically modifying the polymer with an anti-bacterial monomer such as diallyldimethylammonium chloride (DADMAC) (block 440). The monomer may be connected to the polymer of the brush by activating the polymer surface (e.g., applied activation to generate radicals). The monomers, such as DADMAC, may be grafted to the surface by radical polymerization. According to another technique, the polymer surface of the brush is plasma treated to create oxygen functionalities. Co-polymers such as DADMAC co-polymers are then connected to the activated surface. Studies of polyammonium compounds as an anti-bacterial coating are reported in “Ultrathin antibacterial polyammonium coatings on polymer surfaces,” by J. Thome et al, Surface & Coatings Technology, vol. 174-175, September-October 2003, pgs. 584-7.

A second type of coating may be used to chemically modify a polymer-based post-CMP brush to impart a residual bacteria-inhibiting property is a thiocyanate. One technique for coating a post-CMP brush of PVA is to immerse the brush in an aqueous solution of sodium thiocyanate possibly in the presence of an intermediate such as tetrabutylammonium hydroxide (TBAH). One example is to immerse a brush in a solution of sodium thiocyanate (3 moles/dm³) in the presence of TBAH (0.15 moles/dm³) at 80° C. for 5 hours. After immersion, the post-CMP brush may be rinsed and dried. An additional sterilization operation may be performed using ethylene oxide (ETO). Studies of thiocyanate coatings as an anti-bacterial coating on a polymer are reported in “Surface thiocyanation of plasticized poly(vinyl chloride) and its effect on bacterial adhesion,” by N. R. James and A. Jayakrishnan, Biomaterials, v. 24, issue 13, 2003, pgs. 2205-2212.

Block 460 shows another technique to impart a residual bacteria-inhibiting property to a post-CMP brush. In one embodiment, a post-CMP brush of a PVA material is treated with an oxygen-plasma to impart a residual anti-bacterial property to the brush. A study of oxygen-plasma treatment of polymers to provide anti-bacterial coatings are reported in “Adhesion of Pseudomonas aeruginosa strains to untreated and oxygen-plasma treated poly(vinyl chloride) (PVC) from endotracheal intubation devices,” by Triandafillu, K. et al., Biomaterials, v. 24, no. 8, 2003, pgs. 1507-18. The oxygen plasma treatment conditions to modify the polymer surface can be set up at, for example, treatment time of 60 seconds, plasma pressure of 650 mTorr with an oxygen gas flow rate of 900 sccm, and radio-frequency (rf) power of 110 W, as per “CMP of Low-k Methylsilsesquiazane with Oxygen Plasma Treatment for Multilevel Interconnect Applications,” by T. C. Chang, T. M. Tsai, P. T. Liu, C. W. Chen, S. T. Yan, H. Aoki, Y. C. Chang, and T. Y. Tseng, published in Electrochemical and Solid-State Letters, 2004, Vol. 7, No. 6, pp. G122-G124.

A second type of plasma treatment to impart a residual bacteria-inhibiting property to a post-CMP brush is an acetylene plasma immersion ion implantation. One study of this technique is described in “Surface characterization and antibacterial adhesion of poly(ethylene-therephtalate) modified by acetylene plasma immersion ion implantation” by J. Wang et al., Proceedings of 2002 IEEE International Conference on Plasma Science, Piscataway, N.J., IEEE, 2002, pg. 311.

In addition to imparting a residual bacteria-inhibiting property to a post-CMP brush, in another embodiment, a surface energy of a brush may also be modified. Many low-k films tend to have low hydrophobicity. Post-CMP cleaning solutions seek to match the surface energy of the film. It is advantageous if surface energies (that determine hydrophobicity) of the brush surface and post CMP cleaning solutions are close. Certain of the above techniques for imparting a residual anti-bacteria coating also tend to make the surface of a post-CMP brush more hydrophilic. For example, the UV treatment described with reference to block 420, the thiocyanate coating described with reference to block 450, and the oxygen-plasma treatment with reference to block 460 described with reference to in FIG. 4 all tend to make a surface of the post-CMP more hydrophilic. Accordingly, the treatment allows tunable changes of the hydrophobicity of the polymer surface of a post-CMP brush using an appropriate combination of surface treatment parameters, such as UV exposure dose, etc. Some post-CMP solutions used for post-CMP cleaning of low-k dielectric material, may be hydrophobic, organic, or non-organic films. Hydrophobicity can be measured using contact angle measurements, where high values of contact angle (e.g., close to 90° C.) correspond to hydrophobic properties of the surface. Contact angle measurements of some post-CMP clean solutions on a low-k substrate can have contact angles between 60° and 90°. It is desirable to custom treat a post-CMP brush surface by, for example, selecting an appropriate UV dose, to bring brush hydrophobicity to the range of contact angles.

In the preceding paragraphs, specific embodiments are described. It will, however, be evident that various modifications and changes may be made thereto without departing from the broader spirit and scope of the claims. The specification and drawings are, accordingly, to be regarded in an illustrative rather than a restrictive sense.

1. A method comprising:
providing a residual bacteria-inhibiting property to a post-chemical mechanical polish (post-CMP) brush.

2. The method of claim 1, wherein providing a residual bacteria-inhibiting property comprises irradiating the brush with ultraviolet radiation.

3. The method of claim 1, wherein providing a residual bacteria-inhibiting property comprises treating the brush with a plasma.

4. The method of claim 3, wherein the plasma comprises an acetylene plasma.

5. The method of claim 3, wherein the plasma comprises an oxygen plasma.

6. The method of claim 1, wherein providing a residual bacteria-inhibiting property comprises coating the brush with an anti-bacterial monomer.

7. The method of claim 6, wherein coating the brush with an anti-bacterial monomer comprises:
activating a surface of the brush; and
coupling the anti-bacterial monomer to the activated surface.

8. The method of claim 6, wherein the activated monomer comprises diallyldimethylammonium chloride.

9. The method of claim 1, wherein providing a residual bacteria-inhibiting property comprises covalently bonding a thiocyanate moiety to the surface of the brush.

10. The method of claim 1, further comprising modifying a surface energy of the brush.

11. The method of claim 10, wherein modifying the surface energy of the brush comprises modifying to a surface energy similar to a surface energy of a post-CMP cleaning solution.

12. A method comprising:
forming a post-chemical mechanical polish (post-CMP) brush; and
residually modifying a bacteria-inhibiting property of a portion of an exterior surface of the formed brush.

13. The/method of claim 12, wherein residually modifying a portion of the exterior surface comprises irradiating the brush with ultraviolet radiation.

14. The method of claim 12, wherein modifying a portion of the exterior surface of the brush comprises treating the brush with a plasma.

15. The method of claim 14, wherein the plasma comprises one of an acetylene plasma and an oxygen plasma.

16. The method of claim 12, wherein modifying a portion of the exterior surface comprises coating a portion of the exterior surface with an anti-bacterial monomer.

17. The method of claim 16, wherein coating a portion of the exterior surface with an anti-bacterial monomer comprises:
activating a portion of the exterior surface of the brush; and
coupling the anti-bacterial monomer to the activated surface.

18. The method of claim 16, wherein the activated monomer comprises diallyldimethylammonium chloride.

19. The method of claim 12, wherein modifying a portion of the exterior surface comprises covalently bonding a thiocyanate moiety to the surface of the brush.

20. The method of claim 12, further comprising modifying a surface energy of a portion of the exterior surface of the brush.

21. The method of claim 20, wherein modifying the surface energy comprises modifying to a surface energy similar to a surface energy of a post-CMP cleaning solution.

22. An apparatus comprising:
a post-chemical mechanical polish (post-CMP) brush comprising a residual bacteria inhibiting property.

23. The apparatus of claim 22, wherein a portion of an exterior surface of the brush comprises an anti-bacterial monomer.

24. The apparatus of claim 23, wherein the monomer comprises diallyldimethylammonium chloride.

25. The apparatus of claim 22, wherein a portion of an exterior surface of the brush comprises a covalently bonded thiocyanate moiety.

26. The apparatus of claim 22, wherein a portion of an exterior surface of the brush comprises a surface energy similar to a surface energy of a post-CMP cleaning solution.
